(19) United States
(12) Patent Application Publication
(10) Pub. No.: US 2022/0079861 A1
(43) Pub. Date: Mar. 17, 2022

(54) Balanced amino acid formulation

(71) Applicant: IAF NETWORK S.P.A., Brescia (IT)

(72) Inventor: Gabriele TRAPANI, MEDESANO (IT)

(21) Appl. No.: 17/473,254

(22) Filed: Sep. 13, 2021

(30) Foreign Application Priority Data
Sep. 14, 2020 (IT) 102020000021664

(51) Int. Cl. A61K 8/44; A61K 31/198; A61K 31/405; A61K 31/4172; A61K 8/49; A61Q 19/08; A61K 9/00; A23L 33/175; A23C 9/152

(52) U.S. Cl. CPC A61K 8/44; A61K 31/198; A61K 31/405; A61K 31/4172; A61K 8/4946; A23V 2002/00; A61Q 19/08; A61K 9/0056; A23L 33/175; A23C 9/1526; A61K 2800/92; A61K 8/492

(57) ABSTRACT

A balanced formulation based on amino acids, which finds application in the field of functional and antiaging medicine, and in the cosmetic field for the well-being of hair, skin and nails is provided.

### CROSS-REFERENCE TO RELATED APPLICATION

This application is related to and claims the benefit of Italian Patent Application No. 102020000021664 filed Sep. 14, 2020, the entire contents of which are incorporated herein by reference.

### FIELD OF THE INVENTION

The present disclosure finds application in the nutraceutical field and, in particular, in clinical nutrition and functional medicine.

### BACKGROUND OF THE INVENTION

Preparations based on amino acids as food supplements are known.

The need to supplement nutrition with a surplus of amino acids is typical in periods of strong physical stress, such as the period of recovery from an illness, or in periods of intense sporting activity.

The preparations available today follow a standard approach, which includes the nine essential amino acids, although often formulated with a questionable balance from the physiological viewpoint, which does not lead to optimized protein synthesis processes.

Instead in other cases, the proposed amino acid profile is similar to that of red meat, with the same negative and pro-aging effects of methionine.

### SUMMARY OF THE INVENTION

The present disclosure provides a formulation with an optimal amino acid balance, which overcomes the drawbacks of the known formulations.

A first object of the present disclosure is represented by an amino acid preparation.

In other objects of the present disclosure, the preparation is described for use in the nutraceutical and cosmetic field and in a method for improving the aesthetic appearance of hair, skin and nails.

### DETAILED DESCRIPTION

A first object of the present invention is represented by a preparation comprising the following amino acids:
Leucine,
Valine,
Isoleucine,
Lysine,
Phenylalanine,
Threonine,
Methionine,
Histidine,
Cysteine,
Tryptophan, and
Tyrosine.

In an embodiment, no other amino acids are comprised in the preparation of the disclosure.

In a preferred aspect, within the described formulation, leucine, isoleucine and valine are present in a molar ratio of about 2:1:1.

According to an aspect of the present disclosure, the above-listed amino acids are contained in the preparation in an amount of:

| Amino acid | % (g/100 g serving composition) |
|---|---|
| Leucine | 30.76 |
| Valine | 15.38 |
| Isoleucine | 15.38 |
| Lysine | 16.92 |
| Phenylalanine | 2.30 |
| Threonine | 8.46 |
| Methionine | 1.53 |
| Histidine | 3.07 |
| Cysteine | 4.61 |
| Tryptophan | 0.76 |
| Tyrosine | 0.76 |

According to a preferred aspect of the present disclosure, a described preparation comprises:

| Amino acid | amount (mg) |
|---|---|
| Leucine | 2000 |
| Valine | 1000 |
| Isoleucine | 1000 |
| Lysine | 1100 |
| Phenylalanine | 150 |
| Threonine | 550 |
| Methionine | 100 |
| Histidine | 200 |
| Cysteine | 300 |
| Tryptophan | 50 |
| Tyrosine | 50 |

According to a preferred aspect of the present disclosure, the above-listed amino acids are all obtained from fermentation of vegetable origin; thereby, the preparation can also be advantageously taken by or administered to vegetarians and vegans.

According to an aspect of the present disclosure, the described preparation must be taken before and/or after performance, and preferably both before and after, regardless it is a sporting activity or a busy working day.

The assumption is reported in terms of serving, which corresponds to about 100 g of amino acid mixture.

In a preferred aspect, the assumption should occur on an empty stomach to allow better absorption of the amino acid components.

The preparation of the present disclosure may be formulated in powder form, to be dosed appropriately according to need or it may be in the form of swallowable tablets.

The formulation may have further components such as sweeteners and/or flavorings or any additives to increase the solubility of the preparation.

However, it is not excluded that the described preparation may be reformulated as a component of more complex food preparations, such as milk-based drinks, energy bars, etc.

Therefore, such food preparations comprise the preparation of the present disclosure.

In accordance with other objects of the present disclosure, the preparation is described for use in the nutraceutical field.

In particular, such a preparation is described for use in clinical nutrition and functional antiaging medicine.

According to a further aspect of the present disclosure, the described preparation is used in cosmetics.

In particular, the functional preparation of the present disclosure is used for the well-being and tone of skin, for the beauty of hair and for the strength of nails.

According to another object, it is described a method for improving the aesthetic appearance of hair, skin and nails comprising the administration of a preparation of the invention to a subject.

From the above description, the numerous advantages offered by the present disclosure will be immediately apparent to the person skilled in the art.

In particular, the described preparation allows an optimal balance between branched amino acids and essential or “conditionally essential” amino acids.

Thereby, the drawbacks of the currently known preparations are advantageously overcome, in which the imbalance of the ratio between methionine and cysteine results in a pro-aging effect which, in the long term, can lead to homocysteinemia conditions.

The preparation of the present disclosure is therefore an excellent food supplement tool in view of particularly stressful physiological conditions, such as intense and long workouts, conditions of fasting or recovery from diseases, states of ketosis or unbalanced diets, for example lacking carbohydrates.

Furthermore, by virtue of the composition thereof, the preparation of the present disclosure allows to prevent and combat sarcopenia and to confer or increase the anabolic resistance induced by aging.

As reported above, since the amino acids used are all obtained by fermentation of vegetable origin, the preparation of the present disclosure meets the needs of vegetarians and vegans.

1. An amino acid preparation comprising amino acids and other components, wherein said amino acids are represented by:
Leucine,
Valine,
Isoleucine,
Lysine,
Phenylalanine,
Threonine,
Methionine,
Histidine,
Cysteine,
Tryptophan, and
Tyrosine.

2. The amino acid preparation of claim 1, wherein leucine, isoleucine and valine are present in a molar ratio of 2:1:1.

3. The amino acid preparation of claim 1, wherein, in a serving size, corresponding to 100 g, said amino acids are contained in the following amounts:

| | % (g/100) |
|---|---|
| Leucine | 30.76 |
| Valine | 15.38 |
| Isoleucine | 15.38 |
| Lysine | 16.92 |
| Phenylalanine | 2.30 |
| Threonine | 8.46 |
| Methionine | 1.53 |
| Histidine | 3.07 |
| Cysteine | 4.61 |
| Tryptophan | 0.76 |
| Tyrosine | 0.76 |

4. The amino acid preparation of claim 1, comprising:

| Leucine | 2000 | mg, |
|---|---|---|
| Valine | 1000 | mg, |
| Isoleucine | 1000 | mg, |
| Lysine | 1100 | mg, |
| Phenylalanine | 150 | mg, |
| Threonine | 550 | mg, |
| Methionine | 100 | mg, |
| Histidine | 200 | mg, |
| Cysteine | 300 | mg, |
| Tryptophan | 50 mg, and | |
| Tyrosine | 50 | mg. |

5. The amino acid preparation of claim 1, wherein said amino acids are obtained by fermentation of vegetable origin.

6. The amino acid preparation of claim 1, wherein said amino acid preparation is in the form of a powder or swallowable tablets.

7. A food preparation comprising the amino acid preparation of claim 1.

8. The food preparation of claim 7, which is in the form of milk-based drinks or energy bars.

9. A method for providing clinical nutrition and functional antiaging medicine in a subject, said method comprising administering to said subject the amino acid preparation of claim 1.

10. A method for improving aesthetic appearance of hair, skin and nails in a subject, said method comprising administering to said subject the amino acid preparation of claim 1.

11. A method for providing clinical nutrition and functional antiaging medicine in a subject, said method comprising administering to said subject the food preparation of claim 7.

12. A method for improving aesthetic appearance of hair, skin and nails in a subject, said method comprising administering to said subject the food preparation of claim 7.
